(19) United States
(12) Patent Application Publication
(10) Pub. No.: US 2013/0130237 A1
(43) Pub. Date: May 23, 2013

(54) APPARATUS AND METHOD FOR ANALYZING STATE OF DNA

(71) Applicant: CANON KABUSHIKI KAISHA, Tokyo (JP)

(72) Inventor: Toshihiko Ouchi, Machida-shi (JP)

(73) Assignee: CANON KABUSHIKI KAISHA, Tokyo (JP)

(21) Appl. No.: 13/657,780

(22) Filed: Oct. 22, 2012

(30) Foreign Application Priority Data
Nov. 17, 2011 (JP) 2011-251796
Aug. 31, 2012 (JP) 2012-190721

(51) Int. Cl. C12M 1/42; G01N 21/31

(52) U.S. Cl. 435/6.1; 435/287.2

(57) ABSTRACT

The present invention provides an analysis apparatus including: an irradiation section for irradiating the chromatin structure with terahertz waves; a detection section for acquiring a set of terahertz wave spectral information from the chromatin structure; a memory section for memorizing the sets of terahertz wave spectral information corresponding to the states of the chromatin structure; and a data processing section for analyzing the state of the chromatin structure by comparing the set of spectral information acquired in the detection section and the sets of spectral information memorized in the memory section.

### BACKGROUND OF THE INVENTION

1. Field of the Invention

The present invention relates to an apparatus and a method for, for example, analyzing and regulating the states of biological substances such as DNA, mainly by using high frequency electromagnetic waves (hereinafter, also referred to as terahertz waves) of the terahertz band in the region from millimeter waves to terahertz waves (30 GHz or more and 30 THz or less).

2. Description of the Related Art

Techniques and researches for detecting and medically treating diseases such as cancers by observing and controlling the intracellular chromatin structure have been developed.

Chromatin or the chromatin structure means a structure in which DNA is wrapped around histones. Chromatin is classified into two states: one is the state referred to as heterochromatin in which DNA is tightly wrapped around histones and the other is the state referred to as euchromatin in which the wrapping of DNA is loosened. When a gene is expressed, information is read out from the base sequence of DNA in the euchromatin state, and then RNA is produced. Abnormalities such as condensation, coarsening and fine granule formation of chromatin serve as the index for cancer as the cellular abnormalities, and hence it is important to observe such states of abnormalities.

As a method for observing intracellular molecules, methods in which intracellular molecules are labeled with fluorescent dyes are commonly used. However, in DNA and proteins, the control of the labeling positions is difficult. Such labeling sometimes alters the intrinsic functions and properties of biomolecules. Accordingly, such an imaging method that is free from labeling and noninvasive is demanded. Japanese Patent Application Laid-Open No. 2007-216001 discloses an observation of chromatin based on a light scattering method. Japanese Patent Application Laid-Open No. 2007-216001 utilizes an ultrasonic wave because the SN ratio is insufficient when only light is used. In other words, the observation site is irradiated with an ultrasonic wave, and the oscillating component of the ultrasonic wave is superposed on the acquired optical signal as the modulated component of the refractive index of the tissue. Consequently, the sensitivity is improved by synchronous detection. Such a method enables the in-vivo observation (observation as a living organism) instead of observation of a sampled living tissue.

The spectra of the molecules constituting living tissues, in the low-frequency region, namely, the so-called terahertz band (30 GHz or more and 30 THz or less) enable the analysis of the energies corresponding to the skeletal vibrations of the molecules. Accordingly, the terahertz band spectra enable the acquisition of sets of information different from sets of information obtained by infrared spectroscopy about the local vibrational modes between certain intramolecular atoms. Such terahertz band spectra are also referred to as fingerprint spectra, and enable the acquisition of sets of information about the items such as the side chains, the states of functional groups and the steric structures of specific molecules, and allow some molecules themselves to be identified. Japanese Patent Application Laid-Open No. H10-90174 discloses such molecular spectroscopy using terahertz waves; however, the observation of the chromatin structure with the aid of such terahertz wave spectroscopy has never been disclosed.

The imaging using the light scattering technique is a technique to observe the difference in optical refractive index of the tissues. Accordingly, the light scattering technique enables an imaging of cancers based on remarkable structural changes such as the condensation of chromatin, but is hardly capable of monitoring minor changes such as the expression of a gene due to the loosening of chromatin. On the other hand, when a spectroscopic observation is performed by using terahertz waves, the effects of the water absorption in the tissue (loss due to water molecules) and effects of the scattering in the cell membrane usually result in the difficulty in acquiring the sets of spectral information about the chromatin structure from the signals in the terahertz band.

### SUMMARY OF THE INVENTION

The present invention takes as its problem to be solved the provision of a technique enabling the support of the diagnosis of the states of tissues through performing the analysis or the inference of the state of the chromatin structure by using terahertz waves.

The analysis apparatus as an aspect of the present invention includes: an irradiation section for irradiating the chromatin structure with terahertz waves; a detection section for acquiring a set of terahertz wave spectral information from the chromatin structure; a memory section for memorizing the sets of terahertz wave spectral information corresponding to the states of the chromatin structure; and a data processing section for analyzing the state of the chromatin structure through performing a comparison between the set spectral information acquired in the detection section and the sets of spectral information memorized in the memory section.

According to the analysis apparatus as an aspect of the present invention, a comparison between the set of spectral information based on the results detected by using terahertz waves and the sets of spectral information beforehand memorized allows the noninvasive acquisition of the sets of information about the states such as the loosening of the chromatin structure and the condensation of the chromatin structure. Accordingly, by grasping the sets of information about the gene expression, the operations such as the detection of the active state of the cell, the support of the diagnosis of diseases and the regulation of the tissue culture can be performed.

Further features of the present invention will become apparent from the following description of exemplary embodiments with reference to the attached drawings.

### BRIEF DESCRIPTION OF THE DRAWINGS

FIG. 1 is a diagram illustrating the conceptual configuration of the analysis apparatus and the analysis method according to the present invention and the chromatin structure.

FIG. 2 is a chart illustrating the difference of a spectrum in the terahertz band due to the occurrence or the non-occurrence of methylation in a biomolecule.

FIG. 3 is a diagram illustrating the different frequency properties of the scattered terahertz waves due to the difference in the cell size.

FIG. 4 is a diagram illustrating Embodiment 1 of the analysis apparatus and the analysis method according to the present invention.

FIG. 5 is a diagram illustrating Embodiment 2 of the analysis apparatus and the analysis method according to the present invention.

### DESCRIPTION OF THE EMBODIMENTS

Preferred embodiments of the present invention will now be described in detail in accordance with the accompanying drawings.

The features of the present embodiments reside in that the sets of terahertz wave spectral information about the different states of the chromatin structure are beforehand acquired and memorized. A tissue is irradiated with terahertz waves and the reflected and scattered terahertz waves are detected; the resulting set of the terahertz wave spectral information and the memorized sets of spectral information are compared and processed; thus, the state of the chromatin structure is analyzed or inferred. In this way, the sets of information about the loosening of the chromatin structure and the condensation of the chromatin structure are noninvasively acquired. Accordingly, by grasping the sets of information about the gene expression, the operations such as the detection of the active state of the cell, the support of the diagnosis of diseases and the regulation of the tissue culture can be performed.

### Embodiment 1

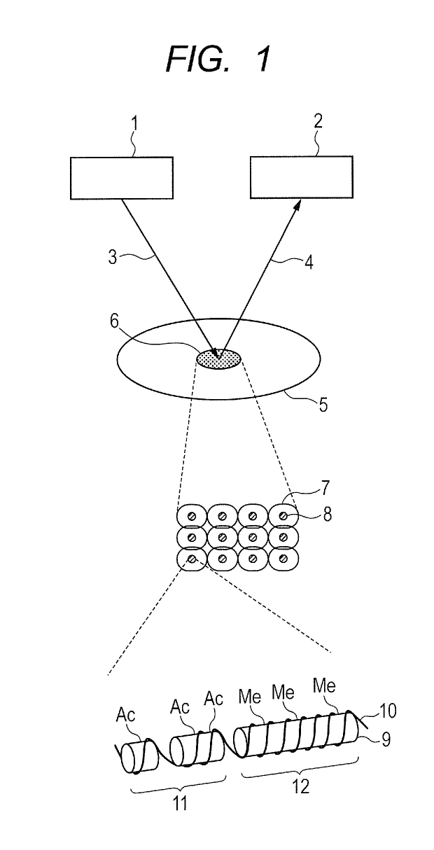

FIG. 1 schematically illustrates the irradiation of a living tissue with terahertz waves, and the reflection of the terahertz waves from the living tissue. The observation area 6 of a living tissue 5 is irradiated with terahertz waves 3 from a terahertz-wave generating section (irradiation section) through the not-shown optical system; the terahertz waves 4 reflected and scattered from the observation area 6 are detected with a terahertz detection section 2 (detection section). The irradiation area is typically about 1 mmφ; however, the size of the irradiation area is not limited to this size when the light condensing system is regulated. As is well known, a living tissue is constituted as an aggregate of a plurality of cells 7 and a plurality of nuclei 8 as shown in the enlarged view of the living tissue 6. DNA 10 is contained in the interior of the nucleus 8, and DNA 10 has a structure in which DNA is wrapped around histones 9. The wrapping of DNA allows DNA to be contained inside the small nucleus. As described in the section of the description of the related art, this wrapped structure includes the loosely wrapped region of euchromatin 11 and the tightly wrapped region of heterochromatin 12. In the region of euchromatin, there is a section allowing the reading out of the base sequence of DNA, and the base sequence is transferred to RNA and a set of information is transferred within the cell. As shown in FIG. 1, in the state of euchromatin, acetyl groups (Ac) are attached to a part of histones, and in the state of heterochromatin, methyl groups (Me) are attached to another part of histones; thus the gene is expressed. Therefore, the knowledge of the proportion of the acetylated state and the knowledge of the proportion of the methylated state enables knowing the degree of activity of the gene information transfer of DNA in the individual cells or the whole cells.

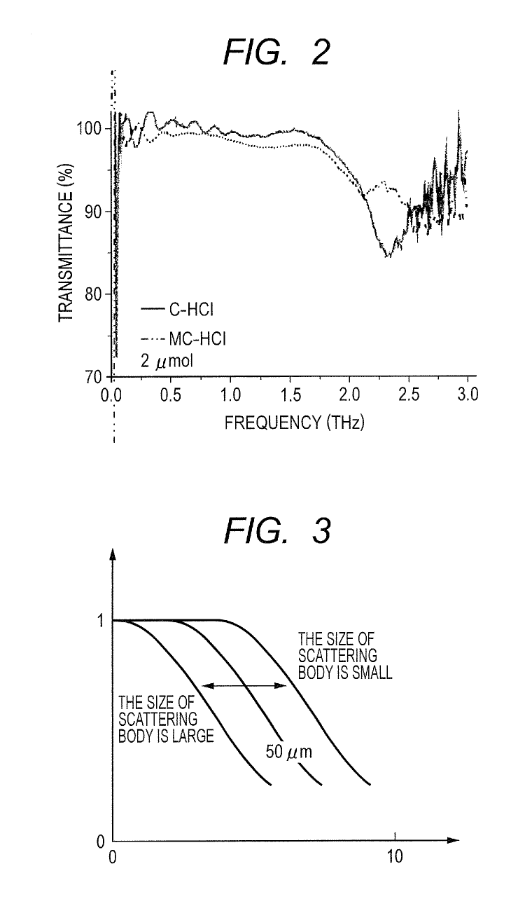

FIG. 2 illustrates a measurement example of the terahertz spectra of cytosine hydrochloride, a base, and methylated cytosine hydrochloride. As can be seen from this figure, methylation results in a significant change of an absorption spectrum in the terahertz region. Acetylation also allows a similar change of an absorption spectrum to be acquired. The terahertz spectra based on the occurrence or nonoccurrence of the attachment of methyl groups or acetyl groups to the specific sites of histones are beforehand acquired, and the acquired spectra are compiled as a database; a comparison of the detected spectrum of a cell to be assayed with the resulting database allows the degree of methylation or the degree of acetylation to be found. The spectral data illustrated in FIG. 2 refers to a spectral data for a single molecule; however, actually, a living tissue is irradiated with terahertz waves, and hence the scattering due to the cell membranes of the cells 7 offers a problem. The size of a cell varies from approximately 1 μm to a few hundreds of microns in various manners, depending on the type of the organism species and the sites. The lipid double layers intervening between the cells and the cells abundant in water are optically different in refractive index, and such a refractive index difference can be a factor inducing the scattering of the terahertz waves. The wavelengths of the employed terahertz waves fall in a range of 10 μm or more and 1000 μm or less, to be just about of the same order of magnitude as the sizes of the cells, and hence the wavelength dispersion due to the Mie scattering and the Rayleigh scattering can occur. FIG. 3 illustrates such a phenomenon. For example, with a scattering body having a size of 50 μm, the effect of the scattering starts to grow from around 2 THz, and the loss of the terahertz waves increases with the increase of the frequency. In other words, the transmittance decreases with the increase of the frequency. Consequently, the actual transmittance is given as a product of the spectrum intrinsic to the molecule in FIG. 2 and the property in the presence of the scattering body of 50 μm in size in FIG. 3. Specifically, due to the effect of the scattering, the transmittance gradually decreases from the baseline set at unity (100%) at around 2 THz to approximately 0.5 (50%) at around 5 THz. In this case, when the size of the scattering body is large, the frequencies exhibiting such an effect are shifted toward the lower frequencies, and when the size of the scattering body is small, the frequencies exhibiting such an effect are shifted toward the higher frequencies.

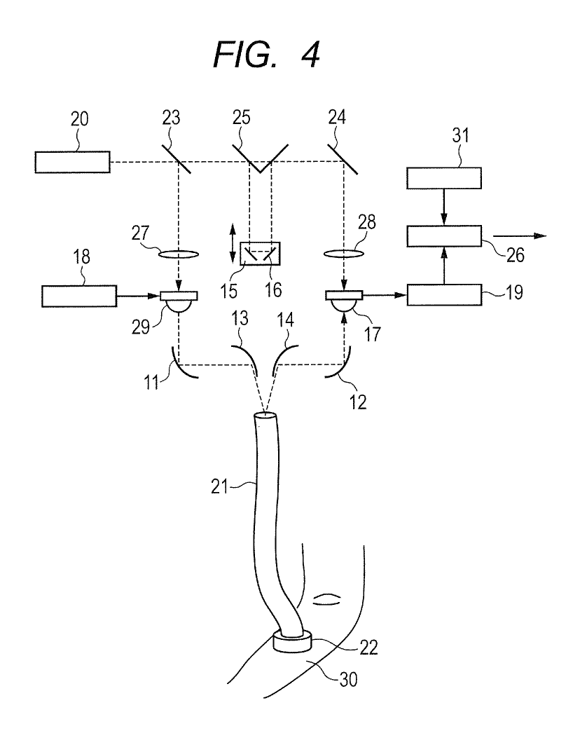

Accordingly, the cell sizes in the measurement sites have to be beforehand examined, and the data processing has to be performed by grasping the properties to be used for correction for each of the sites. The present embodiment is provided with a mechanism in which the cell sizes at the individual sites are compiled as a database, and by selecting the measurement sites, the terahertz spectrum is automatically corrected (correction including the compensation of the loss in the frequency region involving loss). For example, with the scattering body of 50 μm in size, the transmittance gradually decreases from around 2 THz to about half the maximum value at around 5 THz, and hence the actually measured data is compensated with respect to the decrements corresponding to the individual frequencies. When the transmittance at 5 THz is ½ due to the effect of the scattering, the automatic correction as referred to herein means the correction to double the transmittance. The frequency interval for correction is preferably set approximately at the frequency resolution (determined by the displacement magnitude of an optical delay stage 15) determined by the terahertz time region spectroscopic apparatus to be described below. In the actual measurement, as illustrated in FIG. 4, for the purpose of acquiring the reflected pulses as the data, the transmittance is required to be converted into the reflectance; in the case of the terahertz time region spectroscopic apparatus, the calculation involved in the conversion can be performed by a well known method. In this connection, in addition to the correction according to the cell size, the correction data including the loss due to water may be stored and the correction processing with such correction data may be performed in combination with the foregoing correction. In other words, the foregoing spectroscopic apparatus may further be provided with a correction section for correcting the detection results of the detection section with respect to the effect caused by at least one of the loss due to the scattering of the terahertz waves by the lipid double layer constituting the cell membrane corresponding to the cell size in the observation site and the loss due to the water molecules.

FIG. 4 illustrates a specific apparatus configuration for performing the foregoing measurement. A terahertz-wave generating section (irradiation section) includes a structure for generating terahertz pulses by the excitation based on a femto-second laser 20. The laser light beam generated by the femto-second laser 20 such as the ultra-short pulse light beam of 30 fs in pulse width and 1.55 μm in wavelength is bifurcated by a half mirror 23 into two beams; the light beam on the generating section side is focused with a lens 27 as a pumping light beam and a photoconductive element 29 is irradiated with the resulting pumping light beam. The photoconductive element 29 is formed of, for example, low-temperature-grown InGaAs; irradiation of the photoconductive element 29 with a laser pulse under the condition that a voltage is applied to the photoconductive element 29 with a bias supply 18 typically generates an electric field pulse of 200 fs or more and 300 fs or less in pulse width. It should be noted that the material of the element and the type of the element are not limited to the aforementioned material and element, and the aforementioned pulse width is only an instance. The generated terahertz pulse is coupled into a waveguide 21 by two parabolic mirrors 11 and 13, and guided to the other end of the waveguide 21. An observation probe 22 is provided at the other end, and the observation probe 22 is disposed in the vicinity of the tissue 30 to be intended to be measured or the observation probe 22 is brought into contact with the tissue 30 intended to be measured such as a portion of the arm of the human being. The reflected and scattered terahertz pulses from the observation object are again propagated through the waveguide 21 and guided into the photoconductive element 17 by parabolic mirrors 12 and 14. In this way, the propagated terahertz pulses are detected by the detection section. In this case, the other light beam bifurcated from the laser light beam by the half mirror 23 is delayed in time by an optical delay stage 15 and reflection mirrors 16 and also reflected by mirrors 24 and 25, and the photoconductive element 17 serving as a detector is irradiated with the time-delayed light beam through a lens 28. With the system described above, the terahertz time region spectroscopic apparatus is constituted. The detected signals are transmitted through an amplifier 19 to a data processing device 26. In the data processing device 26, the detected signals are compared with the values in the memory device 31 which stores the spectra (the characteristic spectra of chromatin) of the chromatin structure involving the acetylation and the methylation and the sets of information about the cell sizes and the dispersion due to water. Then, the results for determining the state of the chromatin structure are output from the data processing device 26. As described above, the analysis apparatus of the present embodiment includes the irradiation section for irradiation with terahertz waves, the detection section for detecting the terahertz waves, the memory section for memorizing at least the sets of terahertz wave spectral information detected according to the states of the chromatin structure, and the data processing section. The data processing section executes the data processing step in which the state of the chromatin structure is analyzed by comparing the set of spectral information obtained from the detected results of the detection section with the sets of spectral information memorized in the memory section. When the memory section memorizes the characteristic spectra of chromatin respectively based on methylation and acetylation, the data processing section enables the examination of the proportions of euchromatin and heterochromatin, the proportions specifying the state of the chromatin structure.

As the determination results, as described above, for example, the degree of acetylation (the height of the peak of the absorption spectrum when an acetyl group is attached) allows the degree of the loosening of the chromatin (namely, the degree of the activity for the gene expression) of the tissue being examined to be found. The degrees of the state of condensation, the coarsening, the fine granule formation and others (similarly, the dispersion spectra due to these factors are compiled as databases) allow the degree of canceration and others to be found. In the configuration of FIG. 4, a display section (not shown) for displaying the determination results may also be provided. The display section for performing the diagnosis support by displaying the analysis results obtained from the data processing section can display such results by using the characters, coordinates, images and others.

As shown in FIG. 4, when the arm is irradiated with terahertz waves, the state of the affected area on the skin of the arm can be observed. Of course, the application of the concerned method can be applied to the whole body including the face as well as the arm. In the skin cancers (melanoma, non-melanoma), the observation of the state of the chromatin structure allows the canceration range to be inferred. For the diseases other than cancers such as diseases related to the skin including atopic dermatitis, burn, and inflammation, the diagnosis support can be performed by observing the states of the cells of the affected areas. In this case, proteins such as filaggrin and keratin, and the amino acids constituting these proteins play the important roles, and such biomolecules can be observed with the terahertz waves. It is also important that the genes for expressing these proteins function normally, and examinations for the verifying such normal function can be performed. The functions of the prickle cells of the epidermis, the stem cells in the basal stratum of the boundary between the epidermis and the dermis and the fibroblastic cells in the dermis are also important, and accordingly, the observation of DNA for the purpose of examining such functions comes to be important. The use of the probe shown in FIG. 4 as an endoscope to be inserted from the esophagus, trachea or large intestine also enables the observation of the cells of the internal organs. Of course, sampled pathological segments can also be observed.

### Embodiment 2

In Embodiment 1, pulses, namely, broadband terahertz waves are used. However, when the absorption spectrum of the target molecule to be the target is beforehand specified, the two continuous light beams of the two frequencies, namely, the frequency of the spectrum and the frequency to be the reference frequency can be used. For example, as can be seen in the case of such spectra as shown in FIG. 2, when the continuous light beams of 2.1 THz and 2.3 THz are used for irradiation, a comparison of the differences between the signal intensities at the respective frequencies enables the detection of the difference due to the occurrence and non-occurrence of the methylation. Accordingly, in the present embodiment, terahertz waves are generated from such two light sources, for example, as a quantum-cascade laser and a resonant tunneling diode and the tissue is irradiated with such terahertz waves. Then, the reflected light beams and the scattered light beams from the tissue are detected with a terahertz detector such as a microbolometer or a Schottky diode and thus the intensities of the reflected light beams and the scattered light beams are acquired.

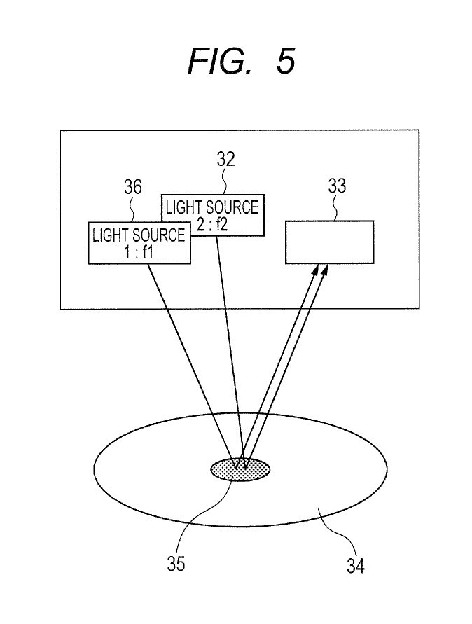

FIG. 5 illustrates an example of the configuration of such an analysis apparatus. Light sources and 2 (36 and 32) are resonant tunneling diodes respectively oscillating at different frequencies f1 and f2, and the same place 35 of a living tissue 34 is irradiated with the terahertz waves based on these oscillations. The reflected light and scattered light from the irradiated place 35 are detected with a detector 33 such as a Schottky barrier diode. Alternate irradiations with the two light sources 1 and 2 allow the detector 33 to detect the intensities at the frequencies f1 and f2 and to obtain the difference between these two intensities. In this way, the proportions of the state 1 of the substance associated with the main absorption frequency f1 and the state 2 of the substance associated with the main absorption frequency f2 can be found. For example, by detecting the proportion of euchromatin, the degree of activity can be determined. In the present embodiment, the devices for generating the terahertz waves and the detection device can be constituted with semiconductor chips, and hence the whole apparatus can be miniaturized and the apparatus can be operated with a low power consumption.

### Embodiment 3

In the foregoing embodiments, the state of the chromatin structure of DNA is observed by signal processing; however, in Embodiment 3, while the state of the chromatin structure of DNA is being observed, the cellular state is regulated or controlled by varying the intensity of the terahertz waves used for irradiation. For example, in the regulation of the degree of chromatin loosening, while the intensity of the terahertz waves is being increased, the absorption intensity of the absorption spectrum is monitored. The power of the terahertz waves at which the absorption intensity change starts to occur is taken as a threshold value, and the proportion of euchromatin is regulated (increased) by further increasing the power from the threshold value. In this way, the degree of activity can be regulated (increased) and the cell differentiation in cell culturing can be regulated (promoted). As a matter of course, the regulations opposite to these regulations can also be performed. In other words, based on the results obtained by the analysis using the aforementioned analysis apparatus or method, by regulating the irradiation power of the terahertz wave having a predetermined frequency used for irradiation of the chromatin structure, the state of the chromatin structure in eukaryotic cells can be regulated. The cell control can also be performed by regulating the irradiation power of the terahertz waves with the aid of this regulation method in such a way that the proportions of euchromatin and heterochromatin satisfy the targeted proportions.

For the regenerative medicine utilizing the method of preparing regenerative tissue controlling cell differentiation, iPS cells and ES cells are suitably used. For such cells, after the sampling, separation and purification, culture for increasing the number of cells to the necessary number of cells is performed. In this case, the observation or the regulation of the state of the chromatin structure by the apparatus or the method according to the present invention can contribute to the efficiency improvement of the regeneration. Moreover, when the differentiation of such cultured cells are induced into targeted tissue cells such as skin, nerve, internal organs, cornea and cardiac muscle, the observation and the control of the state of the chromatin structure with the aid of the apparatus or the method according to the present invention enables the improvement of the yield of the tissue regeneration. In this way, the processing of an object of irradiation with terahertz waves, the object including iPS cells or ES cells as introduced thereinto, with the aid of the aforementioned cell-controlling method, enables the control of the cell differentiation in the object of irradiation.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-251796, filed Nov. 17, 2011, and No. 2012-190721, filed Aug. 31, 2012 which are hereby incorporated by reference herein in their entirety.

What is claimed is:

1. An analysis apparatus comprising:
an irradiation section for irradiating chromatin structure with terahertz waves;
a detection section for acquiring a set of terahertz wave spectral information from the chromatin structure;
a memory section memorizing sets of spectral information corresponding to the states of the chromatin structure; and
a data processing section for analyzing the state of the chromatin structure by comparing the set of spectral information acquired in the detection section and the sets of spectral information memorized in the memory section.

2. The analysis apparatus according to claim 1, further comprising a correction section for correcting the set of spectral information acquired in the detection section, according to the size of the cell, with respect to the effect caused by at least one of the loss due to the scattering of the terahertz waves by the lipid double layer constituting the cell membrane and the loss due to the water molecules involved, wherein the chromatin structure is present in the interior of the cells.

3. The analysis apparatus according to claim 1, wherein in the data processing section, the proportions of euchromatin and heterochromatin are derived as the quantities representing the state of the chromatin structure.

4. The analysis apparatus according to claim 3,
wherein the memory section memorizes the sets of spectral information about methylated chromatin and acetylated chromatin; and
the data processing section determines the proportions of euchromatin and heterochromatin, based on the sets of spectral information about methylated chromatin and acetylated chromatin.

5. The analysis apparatus according to claim 1,
wherein the memory section memorizes a set of dispersion spectral information due to the state of condensation, coarsening or fine granule formation of chromatin; and
the data processing section examines the state of condensation, coarsening or fine granule formation of chromatin by using the dispersion spectrum memorized in the memory section.

6. The analysis apparatus according to claim 1, further comprising a display section for performing support of diagnosis of cancer by displaying the results of the analysis obtained by the data processing section.

7. The analysis apparatus according to claim 1, wherein the irradiation section emits broadband terahertz waves or terahertz waves including continuous light of frequencies associated with the characteristic spectrum of chromatin.

8. The analysis apparatus according to claim 1, wherein the frequencies of the terahertz waves are 30 GHz or more and 30 THz or less.

9. A method for regulating the chromatin structure, comprising:
regulating the state of the chromatin structure by using the analysis apparatus according to claim 1, and by regulating the irradiation power of the terahertz wave of a predetermined frequency used for irradiating the chromatin structure, based on the results obtained by using the analysis apparatus according to claim 1.

10. A cell-controlling method comprising:
regulating the irradiation powers of the terahertz waves by the regulating method according to claim 9, in such a way that the proportions of euchromatin and heterochromatin are targeted proportions.

11. A method for preparing a regenerative tissue comprising:
controlling the cell differentiation in an object to be irradiated with terahertz waves, including the iPS cell or the ES cell as introduced therein, by processing the object to be irradiated by the cell-controlling method according to claim 10.

12. An analysis method comprising:
irradiating the chromatin structure with terahertz waves;
detecting and acquiring a set of terahertz wave spectral information from the chromatin structure;
memorizing sets of terahertz wave spectral information corresponding to the state of the chromatin structure; and
data processing for analyzing the state of the chromatin structure by comparing the set of spectral information acquired in the foregoing detection and the sets of spectral information memorized in the foregoing memorizing operation.
